SEQUENCE LISTING (5 sequences)

SEQ ID NO: 1 - protein, 16 aa, artificial
  Probe
FFFFFGGGRGAGAGAR

SEQ ID NO: 2 - protein, 16 aa, artificial
  Probe
RRRRRGGGRGAGAGAR

SEQ ID NO: 3 - protein, 16 aa, artificial
  Probe
HHHHHGGGRGAGAGAR

SEQ ID NO: 4 - protein, 16 aa, artificial
  Probe
SSSSSGGGRGAGAGAR

SEQ ID NO: 5 - protein, 15 aa, artificial
  Probe
FLLFGGGRGAGAGAR

(19) United States
(12) Patent Application Publication
(10) Pub. No.: US 2021/0278365 A1
(43) Pub. Date: Sep. 9, 2021

(54) MOLECULE DETECTING DEVICE AND MOLECULE DETECTING METHOD

(71) Applicant: KABUSHIKI KAISHA TOSHIBA, TOKYO (JP)

(72) Inventors: Ko YAMADA, Yokohama Kanagawa (JP); Atsunobu ISOBAYASHI, Yokohama Kanagawa (JP); Tatsuro SAITO, Kawasaki Kanagawa (JP)

(73) Assignee: KABUSHIKI KAISHA TOSHIBA, TOKYO (JP)

(21) Appl. No.: 17/017,284

(22) Filed: Sep. 10, 2020

(30) Foreign Application Priority Data
Mar. 5, 2020 (JP) 2020-038036

(51) Int. Cl. G01N 27/414; G01N 1/40; G01N 33/00

(52) U.S. Cl. CPC G01N 27/4141; G01N 33/0031; G01N 1/405

(57) ABSTRACT

According to one embodiment, a molecule detecting device includes a first detector including a first detecting element configured to detect a first molecular group in a gaseous sample, a second detector including a second detecting element configured to detect a second molecular group having a concentration higher than the first molecular group in a gaseous sample, and an absorber including an adsorbing material provided between the first detector and the second detector and configured to adsorb at least the first molecular group.

### CROSS-REFERENCE TO RELATED APPLICATIONS

This application is based upon and claims the benefit of priority from Japanese Patent Application No. 2020-038036, filed Mar. 5, 2020, the entire contents of which are incorporated herein by reference.

### FIELD

Embodiments described herein relate generally to a molecule detecting device and molecule detecting method.

### BACKGROUND

Among gas detecting methods, although various methods are known as methods of detecting a gas constituent having a relatively high concentration, regarding a concentration corresponding to an extremely low concentration, i.e., concentration from ppb to ppt, the detecting methods are limited. Particularly, when a poison gas such as sarin or the like or detonating explosives are detected, the detected substances are kept in a tightly sealed state, and it is the widespread practice to detect imperceptibly diffused volatile constituents (odorous substances) by using a trained police dog or the like. Although the coping method utilizing animals has a remarkable deterrent effect, the method is costly and, in addition, it is difficult to secure constant correctness by the method. Further, unlike a substance simple in chemical structure such as carbon monoxide, the odorous substance has molecular weight of several tens to about 500 and has a complicated chemical structure, and it is difficult to detect the substance by means of a gas sensor configured to detect carbon monoxide or the like.

In order to solve such a problem, a dry-type sensor adopting a carbon nanotube or graphene is proposed. In the dry-type sensor, a probe configured to combine with the odorous substance is fixed on the surface thereof, and the sensor can selectively detect an odorous substance. This probe is constituted of organic molecules in many cases, and is constituted of cells or antibodies derived from a living body in some cases. The probe captures specific odor molecules to make the sensor generate an electric signal, and presence/absence of the object to be detected is confirmed on the basis of a variation in the electric signal.

### BRIEF DESCRIPTION OF THE DRAWINGS

FIG. 1 is a block diagram showing an example of a molecule detecting device of an embodiment.

FIG. 2 is a cross-sectional view showing an example of a detector of the embodiment.

FIG. 3 is a view showing an example of the detector of the embodiment. (a) of FIG. 3 is an enlarged view of a first detecting element, (b) of FIG. 3 is a perspective view of the detector, and (c) of FIG. 3 is an enlarged view of a first probe.

FIG. 4 is block diagram showing an example of the molecule detecting device of the embodiment.

FIG. 5 is a graph showing the constituents of an odor of a mandarin orange.

FIG. 6 is a flowchart showing an example of a molecule detecting method of the embodiment.

FIG. 7 is a cross-sectional view showing an example of the detector of the embodiment in the usage state.

FIG. 8 is a view showing an example of a variation in the current value to be detected by the detecting element of the embodiment.

FIG. 9 is a block diagram showing an example of the molecule detecting device of the embodiment.

### DETAILED DESCRIPTION

In general, according to one embodiment, a molecule detecting device comprises: a first detector including a first detecting element configured to detect a first molecular group in a gaseous sample; a second detector including a second detecting element configured to detect a second molecular group having a concentration higher than the first molecular group in a gaseous sample; and an absorber including an adsorbing material provided between the first detector and the second detector and configured to adsorb at least the first molecular group.

Hereinafter, an embodiment will be described with reference to the accompanying drawings. It should be noted that in the embodiment, parts of the substantially identical configurations are denoted by identical reference symbols and descriptions of the identical parts are partially omitted in some cases. The drawings are schematic views and a relationship between a thickness and planar dimension of each part and ratio between thicknesses of parts, and the like are different from those of the actual parts in some cases.

Molecule Detecting Device

A molecule detecting device according to the embodiment is a device configured to detect molecules in a gaseous sample. As shown in FIG. 1, the molecule detecting device 1 is provided with, for example, a filter device 2, collector 3, detector 4, and recognizer 5.

The filter device 2 is provided with a commonly used medium/high performance filter, and a gaseous sample 6 is passed through the filter by the drive of a fan or pump not shown, thereby removing comparatively large-sized impurities and non-detection object foreign substances from the gaseous sample 6. The collector 3 sends the gaseous sample 6 passing through the filter device 2 to the detector 4. The detector 4 is provided with a detecting element and carries out detection of molecules in the gaseous sample 6. The recognizer 5 is connected to, for example, the detector 4 and determines the type of the gaseous sample 6 on the basis of a measurement value obtained by the detector 4.

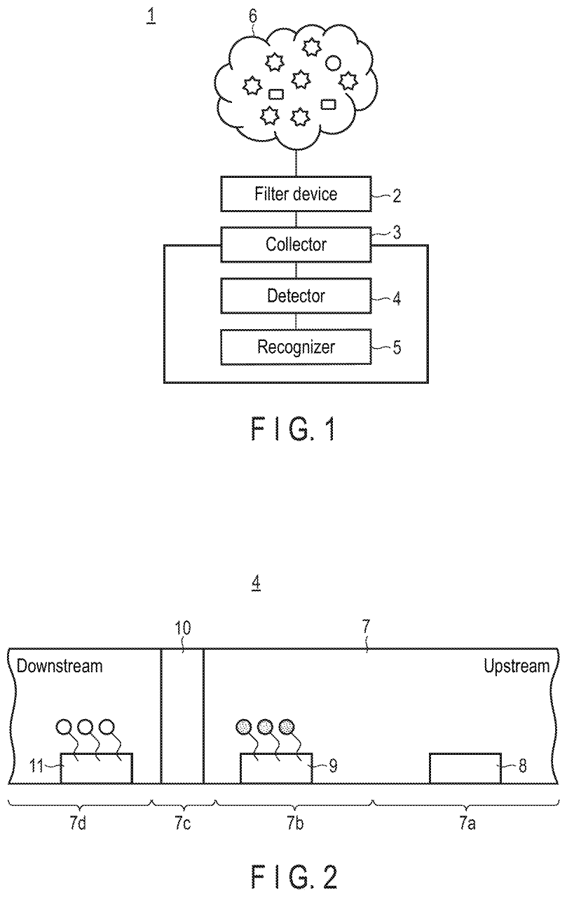

The detector 4 will be described below by using FIG. 2. The detector 4 is provided with a flow path 7 communicating with, for example, the collector 3. The inside of the flow path 7 is divided into four units of, for example, a nonspecific detector 7a, first detector 7b, absorber 7c, and second detector 7d. These four units are arranged in the order mentioned from the upstream side (collector 3 side) of the flow path 7 to the downstream side.

Each of the nonspecific detector 7a, first detector 7b, and second detector 7d is provided with a detecting element configured to detect a molecule in the gaseous sample 6. In the nonspecific detector 7a, a nonspecific detecting element 8 is provided. The nonspecific detecting element 8 is configured to nonspecifically detect a molecule in the gaseous sample 6. The first detector 7b is provided with a first detecting element 9. The first detecting element 9 is configured to detect a first molecular group in the gaseous sample 6. The second detector 7d is provided with a second detecting element 11. The second detecting element 11 is configured to detect a second molecular group in the gaseous sample 6. Here, the second molecular group is a molecular group having a concentration in the gaseous sample higher than the first molecular group. A specific example and determining method of each of the first molecular group and second molecular group will be described later.

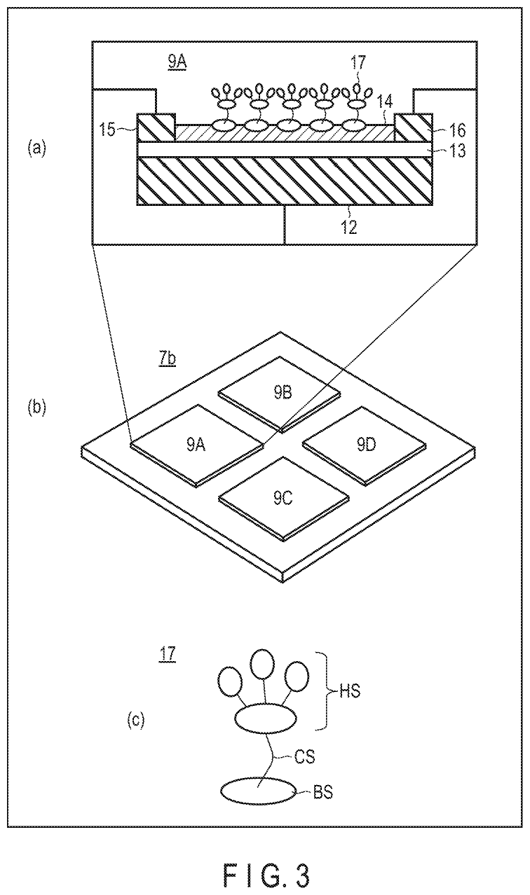

The first detector 7b may be provided with a plurality of first detecting elements 9. Such an example will be described below by using FIG. 3. (b) of FIG. 3 is a perspective view showing an example of the first detector 7b. The first detector 7b is provided with a plurality of first detecting elements 9A to 9D. Although the first detecting elements 9A to 9D can be arranged in, for example, an array form, the detecting elements 9A to 9D are not limited to such an arrangement, and may be arranged on a straight line, may be arranged in a circular form or may be arranged in a random manner. (a) of FIG. 3 shows an enlarged cross-sectional view of the first detecting element 9A which is one of the plurality of first detecting elements 9A to 9D. It is desirable that the first detecting element 9A should have the configuration of a field-effect transistor (FET) provided with a semiconductor substrate functioning as, for example, a gate electrode 12, insulating layer 13 stacked on the gate electrode 12, sensitive membrane 14 which is a channel stacked on the insulating layer 13, source electrode 15 connected to one end of the sensitive membrane 14, and drain electrode 16 connected to the other end of the sensitive membrane 14. On the sensitive membrane 14, a plurality of first probes 17 are fixed.

The material for the gate electrode 12 is, for example, an oxide semiconductor and is, for example, zinc oxide, aluminum oxide, tin oxide, titanium oxide or the like.

It is sufficient if the material for the insulating layer 13 is, for example, SiO₂ to be obtained by thermal oxidation of, for example, the substrate, and the film thickness thereof is adjusted according to the gate voltage to be applied. As the film thickness of the insulating layer 13, a thickness of several tens of nanometers to about 500 nm is selected in many cases.

The material for each of the source electrode 15 and drain electrode 16 is a metal such as gold (Au), silver (Ag), copper (Cu), palladium (Pd), platinum (Pt), nickel (Ni), titanium (Ti), chromium (Cr), aluminum (Al) or the like or conducting substance such as zinc oxide (ZnO), indium tin oxide (ITO), IGZO, conducting polymer or the like.

It is desirable that the sensitive membrane 14 be, for example, a graphene film, carbon nanotube or the like. The sensitive membrane 14 may be constituted of an electric conductor such as gold (Au), silver (Ag), copper (Cu), nickel (Ni), silicon (Si), silicide or the like, or of molybdenum disulfide (MoS₂), tungsten diselenide (WSe₂) or the like.

When graphene is used as the material for the sensitive membrane 14, graphene has the properties as a zero-gap semiconductor, and hence a tendency of a current flow between the source electrode 15 and drain electrode 16 to be generated can also be seen without applying a voltage to the gate electrode 12. Accordingly, the gate electrode 12 need not necessarily be provided.

The first probe 17 is a molecule configured to combine with the first molecular group. In this description, “combination” includes chemical combination and interaction.

It is desirable that the first probe 17 be an organic compound. The organic compound has, for example, a carbonyl group (═O) or amino group (—NH—) as a reaction group for a molecule belonging to the first molecular group. A peptide has a large number of such reaction groups, and hence is favorably used. Regarding the peptide, in addition, the characteristic group possessed by the amino acid further imparts thereto connectivity to the object to be detected. Here, the characteristic group implies a combining group other than hydrogen atoms and combining with the alpha position with which the carbonyl group (═O) or amino group (—NH—) combines. For example, alanine (A) includes a methyl group, valine (V) includes an isopropyl group, and glycine (G) includes hydrogen. By using such an amino group, power of combining with a specific molecule is created.

It is desirable that the first probe 17 be constituted of, as shown in, for example, (c) of FIG. 3, three sites, i.e., an effect site HS having connectivity to the first molecular group, support site BS configured to combine with the sensitive membrane 14, and coupling site CS configured to couple the effect site HS and support site BS to each other. The effect site HS, support site BS, and coupling site CS are each constituted of, for example, a peptide. The amino-acid sequence constituting the peptide of the effect site HS is selected according to the type of the first molecular group. It is desirable that the coupling site CS be a site formed by amino-acid bonding of, for example, a plurality of glycines. It is desirable that the support site BS be a site formed in such a manner that, for example, a glycine and alanine alternately make amino-acid bonding, and arginines combine with the both ends the bonded structure.

For example, the first probe 17 is a peptide having, for example, the following amino-acid sequence.

FFFFF-GGG-RGAGAGAR (sequence number 1)

RRRRR-GGG-RGAGAGAR (sequence number 2)

HHHHH-GGG-RGAGAGAR (sequence number 3)

SSSSS-GGG-RGAGAGAR (sequence number 4)

FLLF-GGG-RGAGAGAR (sequence number 5)

Here, the three parts separated from each other by hyphens are, in order from left to right, the effect site HS, coupling site CS, and support site BS. Alphabets indicate amino acids, and imply the following.

F: phenylalanine, L: leucine, R: arginine, A: alanine, R: arginine, H: histidine, S: serine, and G: glycine

The first probe 17 is not limited to the peptides described above, and may be other molecules. For example, the first probe 17 may be a nucleic acid, other peptide, protein, peptide segment, aptamer, cell or the like.

For example, by applying the first probe 17 dissolved in a solvent to the sensitive membrane 14, it is possible to fix the first probe 17 on the sensitive membrane 14. Further, when the sensitive membrane 14 is exposed to the solution containing therein the first probe 17, the first probes 17 are automatically arranged in such a manner as to cover most of the surface of the sensitive membrane 14 in some cases.

Each of the first detecting elements 9B to 9D may have the configuration identical to the first detecting element 9A described above. However, the first detecting elements 9A to 9D may respectively be provided with first probes 17 of types different from each other. The first probes 17 of the types different from each other may be different from each other in, for example, the action strength (combining strength) on the first molecular group. Thereby, measurement values different from each other can be obtained in the first detecting elements 9A to 9D. However, all the first detecting elements 9A to 9D need not be different from each other, and the action strength of part of the detecting elements may be made different from the other detecting elements.

For example, a plurality of measurement values obtained from the first detecting elements 9A to 9D can be collectively recognized as one pattern. When the types of molecular groups which have come into contact with the detecting element are different from each other, the patterns are also different from each other and, conversely, when the types of molecular groups are identical to each other, identical patterns can be obtained. Accordingly, by acquiring patterns of various already-known molecular groups in advance, and comparing the patterns with the detection results, it is possible to identify the contacted molecular groups. Hereinafter, this method will also be referred to as the “pattern recognition method”.

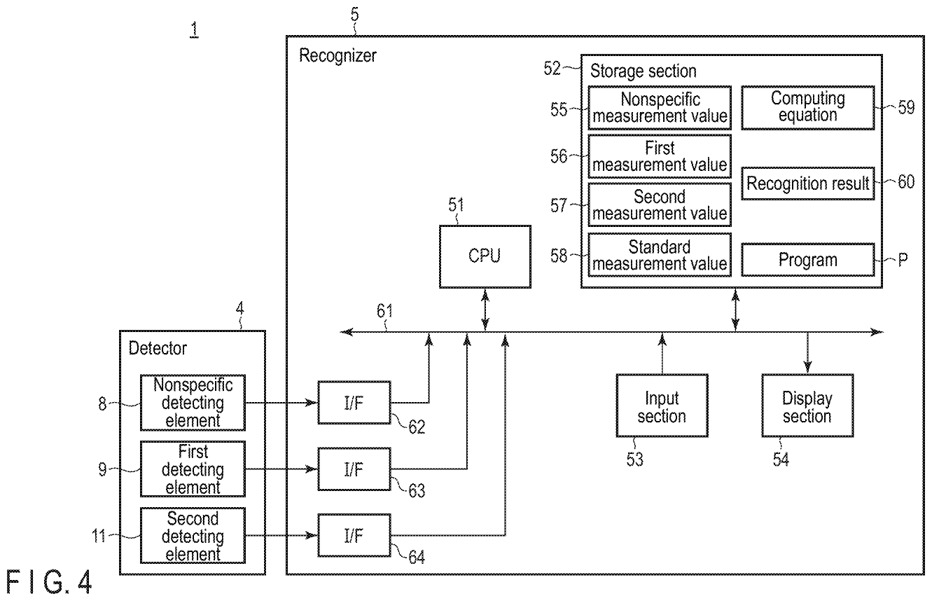

The number of the first detecting elements is not limited to four, and may be, for example, one, and several tens of the first detecting elements may also be provided. The number of the detecting elements is appropriately adjusted according to the necessary number of data items. Although the number of the first detecting element may be one, it is not possible to obtain the detection pattern of an odorous substance by the one first detecting element and the detection accuracy is lowered. Further, even when several hundred detecting elements are provided, the data processing capacity of the recognizer of FIG. 4 is excessively required. The number of the elements is adjusted as the need arises.

The second detector 7d may also be provided with a plurality of second detecting elements 11. The second detecting element 11 can be provided with a second probe configured to combine with the second molecular group in place of the first probe 17 of the first detecting element 9. The second probe may be a probe having a structure shown in (c) of FIG. 3 or may be other organic substances. Although the plurality of second detecting elements 11 may be provided with second probes different from each other, all or part of the second detecting elements 11 may be configured to have identical second probes.

The nonspecific detecting element 8 may have, for example, the configuration identical to the first detecting element 9A, and may be configured in such a manner that the sensitive membrane 14 is not provided with a first probe 17.

The nonspecific detecting element 8, first detecting element 9, and second detecting element 11 are not limited to the configuration of the FET, and may have the configuration of, for example, a surface plasmon resonance (SPR) element, surface acoustic wave (SAW) element, quartz crystal microbalance (QCM) element, microcantilever (MCL) element or the like.

The absorber 7c includes an adsorbing material 10 provided between the first detector 7b and second detector 7d by blocking the flow path 7 as shown in FIG. 2. The adsorbing material 10 is constituted of a material configured to adsorb at least the first molecular group. For example, the adsorbing material 10 is constituted of a porous material. It is desirable that, as the porous material, for example, a carbon material, metal, metallic oxide or the like be used. The porous material may have various forms such as a sheet-like form, pellet-like form, spherical form, form obtained by collecting and unifying nanoparticulate substances or the like.

It is possible to adjust the form, size (volume), and surface area of the porous material according to the type and/or amount (quantity) of the first molecular group. It is desirable that the size (volume) of the porous material be increased or the surface area thereof be increased when the amount (quantity) of the first molecular group is large.

Further, for example, by making a reagent adhere to the surface of the porous material, it is possible to selectively adsorb molecules. It is desirable that the reagent be an organic substance such as a peptide or the like. In the peptide, hydrogen-bonding sites such as NH or the like are contained in large numbers, and hence it is possible to efficiently adsorb the first molecular group by hydrogen bonding. When the porous material is a carbon material, by configuring the peptide serving as the reagent to include the amino-acid sequence such as the support site BS of the aforementioned probe, it is possible to efficiently make the reagent adhere to the porous material. It is desirable that an organic substance containing carboxylic acid be used as the reagent when the porous material is a metal, aggregate or the like of nanoparticles of a metallic oxide. For example, it is also appropriate to use a compound having a fluorinated hydrogen-bonding group such as the compound A shown below as a reagent. When the compound A is used, the carboxylate site is fixed on the surface of the metal or porous material of the metallic oxide, and fluorinated hydrogen-bonding group on the opposite side interacts with the molecules.

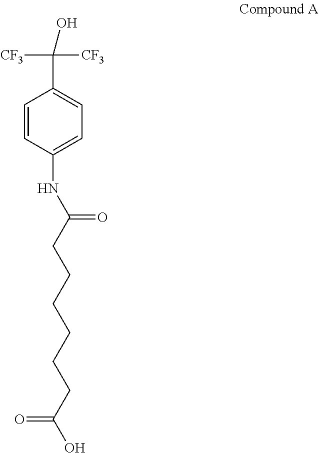 [SMILES: O=C(O)CCCCCCC(=O)Nc1ccc(C(O)(C(F)(F)F)C(F)(F)F)cc1]

The end of the flow path 7 on the downstream side is coupled to, for example, an exhaust port (not shown) communicating with the outside of the molecule detecting device 1.

The recognizer 5 is provided with, for example, a CPU 51, storage section 52, input section 53, and display section 54 as shown in FIG. 4. Although FIG. 4 is a block diagram showing an example of the molecule detecting device, the filter device 2 and collector 3 are omitted.

The CPU 51 controls each part of the molecule detecting device 1 according to a program P stored in the storage section 52, and carries out a mathematical operation for determining the type of the gaseous sample 6 from the measurement result obtained by each detector 4.

The storage section 52 is provided with, for example, a nonvolatile memory and volatile memory, and stores therein a nonspecific measurement value 55 from the nonspecific detecting element 8, first measurement value 56 which is the detection result at the first detector 7b, second measurement value 57 which is the detection result at the second detector 7d, standard measurement value 58 which is the object of comparison, computing equation 59, recognition result 60, program P, and the like.

The input section 53 is provided with, for example, a keyboard, touch panel, button, switch, and the like and inputs parameters and the like necessary for the operation of the molecule detecting device 1. The display section 54 is provided with a display and the like and displays thereon a measurement value and recognition result, and the like.

The CPU 51, storage section 52, input section 53, and display section 54 are connected to each other by, for example, a bus 61. Further, the recognizer 5 may be provided with an interface (I/F) 62 configured to receive an electric signal from the nonspecific detecting element 8 and send the received electric signal to the storage section 52, I/F 63 configured to receive an electric signal from the first detecting element 9 and send the received electric signal to the storage section, and I/F 64 configured to receive an electric signal from the second detecting element 11 and send the received electric signal to the storage section.

The detector 4 may be detachably attached to the molecule detecting device 1. For example, the recognizer 5 and detector 4 may be detachably connected to each other. In that case, the recognizer 5 may be a computer, smartphone, tablet terminal or the like constituted separately from the detector 4.

Determining Method of First Molecular Group and Second Molecular Group

Each of the first molecular group and second molecular group is determined in such a manner that at least one type of molecular group is determined with respect to one type of gaseous sample.

The gaseous sample 6 to be detected by the molecule detecting device of the embodiment is, for example, atmospheric air, exhalation, exhaust gas, gaseous body generated from the substance which is the object to be analyzed, atmospheric air around the substance which is the object to be analyzed or the like. As the substance which is the object to be analyzed, for example, a medicine, food and drink, plants and animals, aromatic goods such as perfume or the like, freight or baggage, household articles or electric appliances, and the like are named.

A molecule to be selected as the first molecular group or second molecular group is, for example, a volatile organic compound (VOC), and is for example, an odorous substance, pheromone substance or the like. Although the target molecule may be, for example, alcohol, ester, aldehyde or the like, the target molecule is not limited to these. The target molecule may be a chemical substance contained in, for example, a drug/stimulant, gunpowder, perishable food, specific plants and animals or the like. The target molecule is, for example, a hydrophobic substance.

The first molecular group can be, for example, molecules which are low in the concentration in the gaseous sample 6 but are important for identifying the type of the gaseous sample 6 or molecules or the like specifically contained in the gaseous sample, and can be an object to be detected for recognizing the gaseous sample 6. The first molecular group may contain therein a plurality of types of molecules having relatively low concentrations in the gaseous sample 6. On the other hand, as the second molecular group, for example, one type of molecules having the highest concentration in the gaseous sample 6 are selected. Alternatively, the second molecular group may contain therein one type or a plurality of types of molecules having a concentration or concentrations relatively high in comparison with other component molecules in the gaseous sample 6. Further, alternatively, the second molecular group may contain therein one type or a plurality of types of molecules being easily detected in addition to being relatively high in concentration.

Although the concentration of the first molecular group in the gaseous sample is not limited, but can be a concentration of, for example, 0.1 ppm to about 1000 ppm or one-tenth to about one-thousandth of the second molecular group. The gaseous sample 6 may contain therein molecules other than the first molecular group and second molecular group.

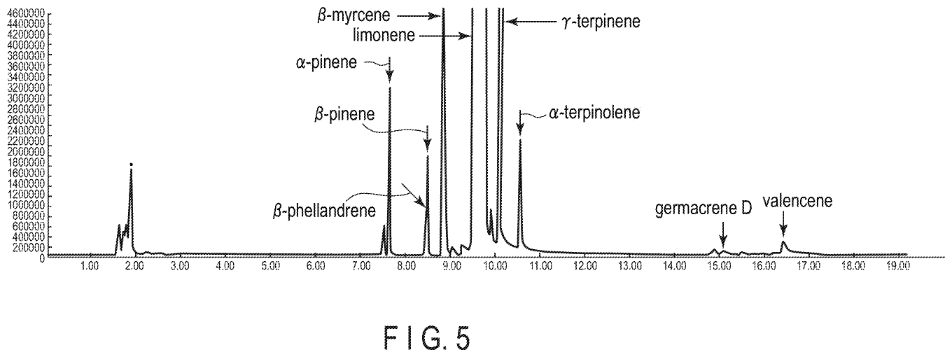

For example, as an example, a case where an odor of a mandarin orange is to be detected will be described below. As the constituents of the molecules contained in the odor of a mandarin orange, as shown in FIG. 5, the amount of each of limonene, β-myrcene, γ-terpinene, and the like is very large, and valencene, germacrene D, and the like are contained as trace constituents. Each of the valencene and germacrene D is one of the specific constituents in the odor of the mandarin orange, and is an important molecule in recognizing the odor of the mandarin orange from other citruses. In such a case, it is possible to select the valencene and/or germacrene D or the like as the first molecular group, and select the limonene, β-myrcene, and/or γ-terpinene as the second molecular group.

When the molecule detecting device 1 configured to detect a specific gaseous sample is to be manufactured, for example, the first molecular group and second molecular group are determined on the basis of the constituents of the gaseous sample, first probe and second probe respectively configured to combine with the first molecular group and second molecular group are designed or selected, detector 4 including the first detecting element 9 and second detecting element 11 respectively provided with the above first probe and second probe is manufactured, and thus the molecule detecting device 1 is assembled.

Accordingly, it is desirable that each of the detectors 4 corresponding to various gaseous samples be manufactured. However, it is also possible to carry out detection of the first molecular group and/or second molecular group by using the same detector 4 and by the pattern recognition method if the gaseous samples similar in constituents to each other are to be detected. Further, a plurality of detectors 4 corresponding to a plurality of gaseous samples 6 may also be arranged in one molecule detecting device 1. Thereby, it is possible to carry out detection with respect to a plurality of types of gaseous samples by means of one molecule detecting device.

Molecule Detecting Method

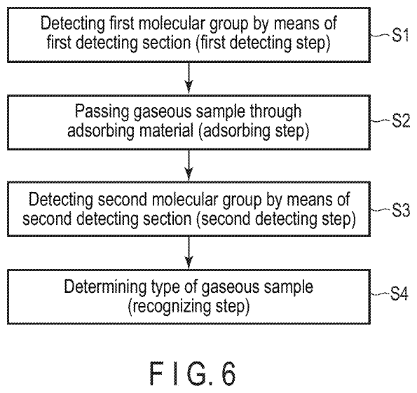

The molecule detecting method using the molecule detecting device 1 described above will be described below. The molecule detecting method includes, for example, the following steps shown in FIG. 6.

(S1) carrying out detection of the first molecular group in the gaseous sample by means of the first detector (first detecting step),

(S2) passing the gaseous sample through the adsorbing material configured to adsorb at least the first molecular group (adsorbing step),

(S3) carrying out detection of the second molecular group in the gaseous sample after being passed through the adsorbing material by means of the second detector (second detecting step), and

(S4) determining the type of the gaseous sample from the results of detection of the first molecular group and detection of the second molecular group (recognizing step).

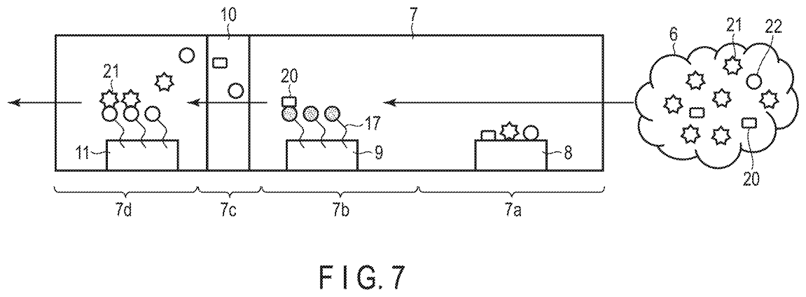

Hereinafter, detailed procedures of the molecule detecting method will be described by using FIG. 7.

The gaseous sample 6 contains therein the first molecular group including a plurality of types of molecular groups, for example, the first molecular group including the first molecules 20 and second molecular group including the second molecules 21 greater in number than the first molecules 20. Here, molecules other than the first molecular group and second molecular group are collectively referred to also as non-detection object molecules 22.

First, the molecule detecting device 1 provided with the detector 4 corresponding to the type of the gaseous sample 6 desired to be detected is prepared. The molecule detecting device 1 is made close to the gaseous sample 6, and by driving the fan of the collector 3, the gaseous sample 6 is sent to the filter device 2, whereby the foreign substances are removed. It is also acceptable to remove the unnecessary foreign substances in the gaseous sample 6 by further ionizing the molecules contained in the gaseous sample 6 to thereby divide the molecules in terms of mass after passing the gaseous sample 6 through the filter device 2. Thereafter, the gaseous sample 6 is introduced into the collector 3, and is made to flow into the flow path 7. Inside the flow path 7, the gaseous sample 6 is passed through the nonspecific detector 7a and first detector 7b in the order mentioned, and is brought into contact with the nonspecific detecting element 8 and first detecting element 9 in sequence. Further, detection of molecules is carried out by each detecting element.

When each of the detecting elements constitutes the FET shown in (a) of FIG. 3, a fixed voltage is applied to, for example, the gate electrode 12, further a voltage is applied between the source electrode 15 and drain electrode 16, and a value of a current flowing between the source electrode 15 and drain electrode 16 is measured.

The nonspecific detecting element 8 nonspecifically detects the first molecules 20, second molecules 21, and non-detection object molecules 22, and hence a measurement value obtained by adding up values of these types of molecules is acquired. For example, it can be seen from this measurement value that the gaseous sample 6 has been introduced into the flow path 7. The measurement to be carried out by the nonspecific detecting element 8 need not necessarily be carried out and, in that case, the molecule detecting device 1 may not be provided with the nonspecific detector 7a. Alternatively, the first detector 7b may be provided with a plurality of first detecting elements 9, one of the first detecting elements 9 may be configured to be provided with no first probe 17 and may be used as the nonspecific detecting element 8.

In the first detecting element 9, a measurement value associated with the first molecular group is obtained (first detecting step S1). For example, in the case where the first detecting element 9 shown in (a) of FIG. 3 is used, when the first molecules 20 come close to the first probe 17, the first molecules 20 are attracted to the first probe 17 by the force or the like of hydrogen bonding, and are brought into contact with the first probe 17 depending on the circumstances. As a result, an exchange of electrons occurs between the first molecules 20 and first probe 17 and the exchange of electrons appears as an electric change in the sensitive membrane 14. The electric change causes the value of a current flowing between the source electrode 15 and drain electrode 16 to change, and hence by measuring the current value, it is possible to detect combining of the first molecules 20.

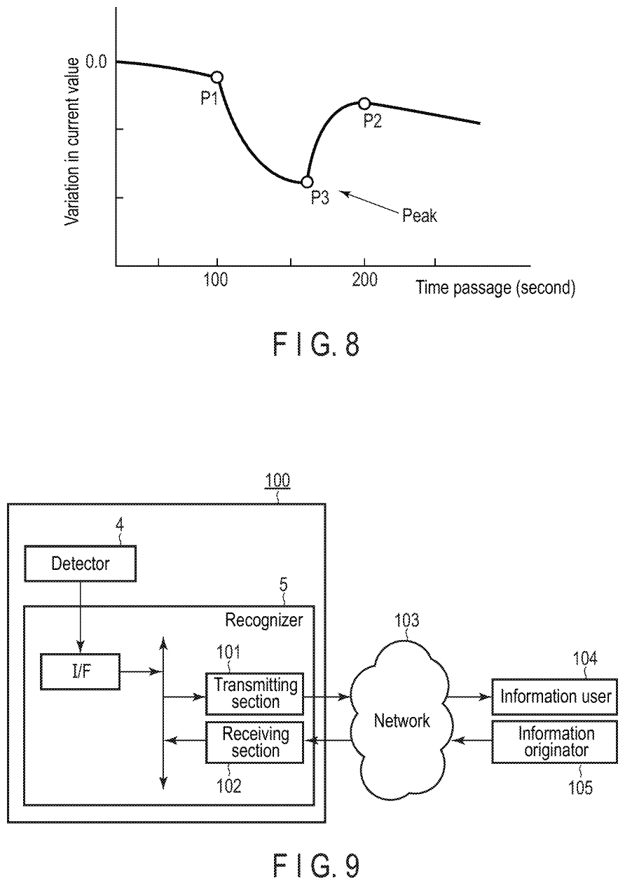

For example, when a graph in which the axis of abscissas indicates time passage and axis of ordinate indicates variation in current value as shown in FIG. 8 is prepared, after the contact of the first molecular group, a variation (for example, lowering of the current value due to an increase in the resistance) in the current value occurs. Thereafter, the molecules gradually separate from the first probe 17, whereby the current value gets closer to the original value. For example, the area surrounded by the lines connecting between the current value P1 at the point of time when the variation in the current value has started, current value P2 at the point of time when the variation has ended, and current value P3 which is the tip of the variation amount may be made the measurement value.

Thereafter, the gaseous sample 6 passes through the adsorbing material 10 (passing step S2). While the gaseous sample 6 passes through the adsorbing material 10, the adsorbing material 10 adsorbs at least the first molecular group. Accordingly, the first molecules 20 adhere to the adsorbing material 10 and remain therein, and molecules which have not been adsorbed flow into the second detector 7d. Accordingly, the gaseous sample 6 before passing through the adsorbing material 10 contains therein the first molecular group, second molecular group, and non-detection object molecules 22, and gaseous sample 6 after passing through the adsorbing material 10 can contain therein the second molecular group and non-detection object molecules 22. However, the gaseous sample 6 after passing through the adsorbing material 10 need not necessarily contain therein totally no first molecules 20, and the first molecules 20 may flow into the second detector 7d in an amount less than the detection limit. Further, part of the second molecules 21 may adhere to the adsorbing material 10 and remain therein. All or part of the non-detection object molecules 22 may adhere to the adsorbing material 10 and remain therein.

After passing through the adsorbing material 10, the gaseous sample 6 enters the second detector 7d and comes into contact with the second detecting element 11. In the second detecting element 11, a measurement value concerning the second molecular group including the second molecules 21 is obtained (second measuring step S3). The measurement is carried out in the method identical to the method described in connection with the first detecting element 9.

The measurement values obtained from the nonspecific detecting element 8, first detecting element 9, and second detecting element 11 are stored in the storage section 52 respectively as the nonspecific measurement value 55, first measurement value 56, and second measurement value 57. The first measurement value 56 may be a first pattern obtained by adding up measurement results from a plurality of first detecting elements 9. Likewise, the second measurement value 57 may be a second pattern obtained by adding up measurement results from a plurality of second detecting elements 11.

Next, in the recognizer 5, the type of the gaseous sample 6 is determined from the measurement results obtained by the first detector 7b and second detector 7d (recognizing step S4).

The first detector 7b aims at detecting the first molecular group, and hence if the most part of the gaseous sample 6 is the first molecular group, it is possible to use the result at the first detector 7b as the result of the first molecular group, and by using the result, it is possible to determine the type of the gaseous sample 6. However, actually, various molecules are contained in the gaseous sample 6 and, as in the case of this molecule detecting method where molecules of an amount extremely small for an object to be detected are selected as the first molecular group, the second molecular group of a very high concentration in comparison with the first molecular group is also contained in many cases. The second molecular group nonspecifically combines with the first probe 17 of the first detecting element 9 or sensitive membrane 14 in some cases. In that case, the measurement value becomes a value obtained by adding up the measurement value of the first molecular group and that of the second molecular group, and it becomes difficult to recognize the gaseous sample 6 by only the result of the first detector 7b. At this time, the amount of the second molecular group which has combined with the first detecting element 9 is small relatively to the whole amount of the second molecular group, and hence a sufficient amount of the second molecular group exists in the second detector 7d after passing through the adsorbing material 10, and is detected also in the second detector 7d.

For example, in the case where the detecting element is an FET, when the second molecular group has hardly combined with the first detector 7b, the first measurement value can be obtained as a variation (amount of change) in the current value less than the second measurement value. However, when the second molecular group has also combined with the first detector 7b, the first measurement value can become a mixed value obtained by adding up both the measurement value the first molecular group and that of second molecular group. For example, the first measurement value in the first detector 7b can become an amount of variation greater than the second measurement value.

In such a case, for example, by subtracting the second measurement value 57 from the first measurement value 56, the influence of the second molecular group on the first measurement value 56 is removed, and it is possible to more accurately obtain the measurement value of the first molecular group. Accordingly, when the non-detection object molecules which easily combine with the first detecting element 9 and are easily detected by the first detecting element 9 are made apparent, it is also effective to select the non-detection object molecules as a second molecular group. The above mathematical operation is carried out by the CPU 51 of the recognizer 5. For example, the CPU 51 takes out the first measurement value 56, second measurement value 57, and computing equation 59 from the storage section 52, and carries out the mathematical operation by using these values and equation.

For example, when a plurality of first detecting elements 9 and plurality of second detecting elements 11 are used, it is sufficient if each of second detecting elements 9 corresponding to each specific first detecting element 9 is determined, and a subtraction operation is carried out between each pair of the first and second detecting elements in the correspondence relationship. The measurement values after the subtraction may be added up and the resultant value may be used as one pattern of the first molecular group.

When no influence of the second measurement value on the first measurement value is found, the first measurement value may be used as it is as the result of the first molecular group.

The type of the gaseous sample 6 is determined from the measurement value or pattern of the first molecular group obtained in the manner described above. For example, the measurement value or pattern of the first molecular group is compared with the measurement value or pattern (standard measurement value 58) of the already-known standard sample stored in the storage section 52 by the CPU 51 and, when the compared values or patters are similar to each other, it is possible to determine that the gaseous sample 6 is of the same type as the standard gaseous sample. When the values or patterns are not similar to each other, it is possible to repetitively carry out this comparison by using a plurality of types of standard measurement values 58 to thereby find out a standard gaseous sample having a measurement value or pattern similar to the measurement value or pattern of the first molecular group. For the recognition of the type of the gaseous sample 6, the measurement result of the second molecular group may also be used in the same manner in addition to the measurement value of the first molecular group.

The gaseous sample 6 which has passed through the second detector 7d is discharged to the outside of the device. In order to render the gaseous sample 6 harmless, it is possible to provide the exhaust port with an adsorbing substance such as zeolite or metal organic framework (MOF), e.g., HKUST-1. Further, when the gaseous sample 6 is constituted of ingredients readily dissolving in water, it is also effective to dispose of the gaseous sample 6 by dissolving the sample 6 in water (bubbling in water).

As has been described above, according to the molecule detecting device and molecule detecting method following the embodiment, it is possible to detect the gaseous sample 6 with a high degree of sensitivity. Particularly, even when the low-concentration first molecular group and high-concentration second molecular group are coexistent with each other, by using the first detector 7b, absorber 7c, and second detector 7d, it is possible to obtain the more accurate detection value of the first molecular group. Thereby, it is possible to detect various types of gaseous samples 6 with a higher degree of sensitivity.

The molecule detecting device of the embodiment may also be configured to communicate with the outside through an information network. Such a molecule detecting device 100 will be described below by using FIG. 9. Although FIG. 9 is a block diagram showing an example of the molecule detecting device, the filter device 2 and collector 3 are omitted therefrom. The recognizer 5 of the molecule detecting device 100 is further provided with a transmitting section 101 and receiving section 102. Although the recognizer 5 is provided with, as in the case of the recognizer 5 shown in FIG. 4, a CPU 51, storage section 52, input section 53, and display section 54, these members are omitted in FIG. 4.

The transmitting section 101 is connected to an information network 103. The transmitting section 101 has a function of taking out the measurement values such as the nonspecific measurement value 55, first measurement value 56, second measurement value 57, and the like and/or recognition result 60 of the gaseous sample from the storage section, and transmitting the values and/or result to a terminal of an information user 104 through the information network 103. The terminal of the information user 104 can be, for example, a personal computer, smartphone, tablet or the like. Alternatively, the terminal may be a molecule detecting device of the other embodiment, other analyzing devices or the like.

The receiving section 102 has a function of receiving information transmitted from a terminal of an information originator 105 through the information network 103 and storing the received information in the storage section 52. The terminal of the information originator 105 can be, for example, a personal computer, smartphone, tablet or the like. Alternatively, the terminal may be a molecule detecting device of the other embodiment, other analyzing devices or the like. The information transmitted from the information originator 105 is, for example, a standard measurement value 58 obtained from an already-known standard gaseous sample. The CPU 51 uses the obtained standard measurement value 58 for comparison with the measurement value of the gaseous sample 6. The information network 103 may be, for example, the Internet or may be intranet.

As described above, by acquiring information from an external information network and referring to the information, a large number of information items need not be stored in the device and can be replaced with the external information, and hence it is possible to further downsize the molecule detecting device 1 and enhance the portability thereof. Furthermore, it is also possible to instantly acquire a plurality of standard measurement values 58. For example, a method of using the device contrived in such a manner that the molecule detecting devices 1 are arranged at a plurality of desired places in advance, obtained data is collected from each place and is analyzed, and the resultant data items are utilized for evacuation guidance at the time of abnormal state occurrence is also enabled.

### EXAMPLES

Hereinafter, examples in which the molecule detecting device and molecule detecting method of the embodiment are used and evaluation results of the examples will be described.

### Example 1 Analysis of Gaseous Sample Using Molecule Detecting Device

Manufacture of Detecting Element

First, a detecting element obtained by combining a GrFET and probe was prepared in the following manner. A graphene film was formed by transferring graphite to the substrate by the graphite exfoliating method or by growing a graphene film on the surface of a metal by means of the chemical vapor deposition (CVD). The graphene of a single layer or a plurality of layers grown on the surface of the metal was transferred to a polymer film, and the transferred graphene was transferred again to a semiconductor substrate for manufacturing a desired field effect transistor (FET). For example, a graphene film was formed on a surface of copper foil by the CVD of making methane gas flow along the surface of the copper foil under a temperature condition of about 1000° C.

Next, the graphene film formed on the surface of the copper foil was coated with a polymethylmethacrylate film by using the spin coating method at a rotational speed of 4000 rpm, the copper foil film on the reverse side was etched by using an ammonium persulfate solution having a concentration of 0.1 M, and the graphene film floating on the solution was retrieved. Thereby, the graphene film was transferred to the polymethylmethacrylate film side. The surfaces of the films were thoroughly washed and thereafter the resultant matter was transferred again to the silicon substrate. The excess polymethylmethacrylate film was removed by dissolving in acetone. The graphene transferred to the silicon substrate was coated with a resist and was patterned, thereby forming a pattern having an electrode interval of 10 μm by means of oxygen plasma. An FET structure provided with a source electrode and drain electrode was formed by depositing electrodes. An FET type sensor structure in which the graphene is arranged on an oxide film formed on the surface of the silicon substrate, the graphen is interposed between the source electrode and drain electrode, and the silicon substrate side thereof is made the gate electrode was formed. The order of these steps may be reversed. That is, the electrodes may be formed first and thereafter the graphene may be transferred.

Four pieces of GrFET elements were manufactured, and probes different from each other were provided on the surface of the graphene. As the probes, a probe RR (arrangement number 2), probe HH (arrangement number 3), and probe SS (arrangement number 4) which are shown in Table 1 were used.

TABLE 1

| Detecting | Probe | Effect | Coupling | Support |
|---|---|---|---|---|
| element | name | site HS | site CS | site BS |
| First | Probe RR | RRRRR | GGG | RGAGAGAR |
| detecting | | | | |
| element A | | | | |
| First | Probe HH | HHHHH | GGG | RGAGAGAR |
| detecting | | | | |
| element B | | | | |
| First | Probe SS | SSSSS | GGG | RGAGAGAR |
| detecting | | | | |
| element C | | | | |
| First | — | — | — | — |
| detecting | | | | |
| element D | | | | |

Each of the probes was dissolved in a methanol solution at a concentration of 10 nM, and the sensitive membrane was immersed therein for several minutes to be fixed. Alternatively, each of the probes was dissolved in a phosphoric acid-based buffer solution, and the sensitive membrane was immersed therein for several minutes to be set. Thereby, first detecting elements A to C each provided with probes different from each other were obtained. The first detecting element D was provided with no probe, and was brought into a state where the graphene surface was exposed. These detecting elements were placed in the flow path.

Adjustment of Gaseous Sample

A mixed gas X prepared by mixing an odor A, odor B, and odor C of Table 2 was prepared. The odor A, odor B, and odor C are each constituents contained in mandarin oranges belonging to the citrus family.

TABLE 2

| | | | Vapor | |
|---|---|---|---|---|
| | Odor | Molecular | pressure | |
| | molecule | weight | (Pa) | Origin |
| Odor A | D-(+)-Limonene | 136 | 205.3 | Mandarin |
| | | | | orange |
| Odor B | Valencene | 204 | 1.5 | Mandarin |
| | | | | orange |
| Odor C | Germacrene D | 204 | 0.9 | Mandarin |
| | | | | orange |

As the odors A to C, saturated vapors produced in bottles were utilized. The vapors were taken out by using syringes, and the vapors were put into a Tedlar bag, whereby a mixed gas X obtained by mixing the three odors was produced.

Detection of Gaseous Sample

The mixed gas X was poured into the flow path from the collection section, was made to reach the first detector placed at the upstream part, and then measurement was carried out for each of the first detecting elements A to D. The results are shown in Table 3. The results are to evaluate the strengths of the first detecting elements by using the first detecting element D as the reference.

TABLE 3

| Detecting | Probe | |
|---|---|---|
| element | name | Strength |
| First | Probe RR | 5 |
| detecting | | |
| element A | | |
| First | Probe HH | 3 |
| detecting | | |
| element B | | |
| First | Probe SS | 0 |
| detecting | | |
| element C | | |
| First | None | 5 |
| detecting | (exposure of | |
| element D | graphene surface) | |

Next, the mixed gas X was passed through a porous body (adsorbing material). As the porous body, a porous body adopting 2,6-Diphenyl-p-phenylene Oxide known as TenaxTA as a base was used. The surface area of the porous body was 35 m²/g, and diameter of the hole was about 200 nm. Then, TenaxTA in the form of beads was supported on a meshed polyethylene sheet having an aperture ratio of about 30% and was placed at a downstream part of the first detector. On the further downstream part of the porous body, the second detecting elements A to D were placed.

The second detecting elements A to D were manufactured in the following manner. Four pieces of elements identical to the aforementioned GrFET elements were prepared, the probe FL (arrangement number 5) shown in Table 4 was provided on the surface of the graphene of each of three of the four GrFET elements, and one of the four GrFET elements was provided with no probe and was brought into a state where the graphene surface was exposed. The probes were fixed in the manner identical to the first detecting element.

TABLE 4

| Detecting | Probe | Effect | Coupling | Support |
|---|---|---|---|---|
| element | name | site HS | site CS | site BS |
| Second | Probe FL | FLLF | GGG | RGAGAGAR |
| detecting | | | | |
| element A | | | | |
| Second | Probe FL | FLLF | GGG | RGAGAGAR |
| detecting | | | | |
| element B | | | | |
| Second | Probe FL | FLLF | GGG | RGAGAGAR |
| detecting | | | | |
| element C | | | | |
| Second | — | — | — | — |
| detecting | | | | |
| element D | | | | |

With respect to the second detecting elements A to D, measurement of the mixed gas X after passing through the adsorbing material was carried out. Measurement results were as shown in Table 5.

TABLE 5

| Detecting | Probe | |
|---|---|---|
| element | name | Strength |
| Second | Probe FL | 5 |
| detecting | | |
| element A | | |
| Second | Probe FL | 5 |
| detecting | | |
| element B | | |
| Second | Probe FL | 5 |
| detecting | | |
| element C | | |
| Second | None | 3 |
| detecting | (exposure of | |
| element D | graphene surface) | |

### Example 2 Study on Porous Material

Manufacture of Adsorbing Material A and Adsorbing Material B

First, 5 mL of an aqueous solution in which 0.1 g of zinc oxide nanoparticles (manufactured by Aldrich, diameter 50 nm or less) were dispersed is prepared. The nanoparticles are dispersed in water by utilizing an ultrasonic wave. The compound A or compound B shown below was put into a flask and was dissolved in 5 mL of ethanol or dimethyl sulfoxide (DMSO) each serving as a water-soluble solvent.

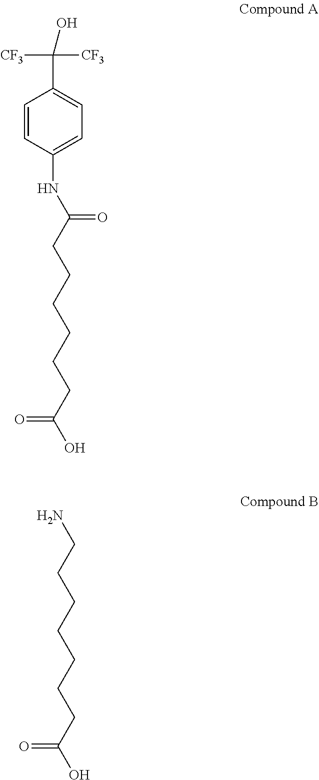 [SMILES: O=C(O)CCCCCCC(=O)Nc1ccc(C(O)(C(F)(F)F)C(F)(F)F)cc1 ; NCCCCCCCC(=O)O]

Both the above solutions were mixed with each other and stirred. Stirring of the mixed solution was continued while keeping the dispersed state of the nanoparticles at intervals by means of the ultrasonic wave. After terminating the stirring, the solid substance was taken out by filtration. The solid substance was thoroughly washed with ethanol or methanol, and was retrieved. As a result, the nanoparticles A to which the compound A was attached and nanoparticles B to which the compound B was attached were obtained. The nanoparticles A or nanoparticles B were dispersed again in ethanol by using the ultrasonic wave, the ethanol in which the nanoparticles A or B were dispersed was applied to a meshed fluorine resin (PTFE) and was thoroughly dried. In this way, the adsorbing material A and adsorbing material B were obtained.

Detection of Gaseous Sample

The adsorbing material A or adsorbing material B was arranged between the first detecting elements A to D and second detecting elements A to D all of which are identical to the example 1, and detection was carried out at each of the second detecting elements A to D. The results of evaluating the strengths of the second detecting elements by using the second detecting element D as the reference are shown in Table 6 and Table 7.

TABLE 6
Adsorbing material A

| Cell | Probe | |
|---|---|---|
| position | name | Strength |
| Second | Probe FL | 3 |
| detecting | | |
| element A | | |
| Second | Probe FL | 3 |
| detecting | | |
| element B | | |
| Second | Probe FL | 3 |
| detecting | | |
| element C | | |
| Second | None | 1 |
| detecting | (exposure of | |
| element D | graphene surface) | |

TABLE 7
Adsorbing material B

| Cell | Probe | |
|---|---|---|
| position | name | Strength |
| Second | Probe FL | 4 |
| detecting | | |
| element A | | |
| Second | Probe FL | 4 |
| detecting | | |
| element B | | |
| Second | Probe FL | 4 |
| detecting | | |
| element C | | |
| Second | None | 2 |
| detecting | (exposure of | |
| element D | graphene surface) | |

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A molecule detecting device comprising:
a first detector including a first detecting element configured to detect a first molecular group in a gaseous sample;
a second detector including a second detecting element configured to detect a second molecular group having a concentration higher than the first molecular group in the gaseous sample; and
an absorber including an adsorbing material provided between the first detector and the second detector and configured to adsorb at least the first molecular group.

2. The device of claim 1, wherein
the first detecting element includes a first probe configured to combine with the first molecular group, and
the second detecting element includes a second probe configured to combine with the second molecular group.

3. The device of claim 2, wherein
the first probe and/or the second probe are constituted of an organic substance.

4. The device of claim 3, wherein
the first probe and/or the second probe are constituted of a peptide.

5. The device of claim 1, further comprising a recognizer configured to determine a type of the gaseous sample from measurement results obtained by the first detector and the second detector.

6. The device of claim 5, wherein
the recognizer is configured to determine the type of the gaseous sample by using an operation result to be obtained by subtracting the measurement result of the second detector from the measurement result of the first detector.

7. The device of claim 2, further comprising a recognizer configured to determine the type of the gaseous sample by using a first pattern and a second pattern, wherein
the first detector includes a plurality of first detecting elements each including first probes of types different from each other, and the first pattern is formed by adding up measurement results from the plurality of first detecting elements, and
the second detector includes a plurality of second detecting elements each including second probes of types different from each other, and the second pattern is formed by adding up measurement results from the plurality of second detecting elements.

8. The device of claim 2, wherein
the first detecting element includes a graphene film possessing a surface on which the first probe is fixed, a source electrode connected to one end of the graphene film, and a drain electrode connected to the other end of the graphene film, and
the second detecting element includes a graphene film possessing a surface on which the second probe is fixed, a source electrode connected to one end of the graphene film, and a drain electrode connected to the other end of the graphene film.

9. The device of claim 1, wherein
the adsorbing material is constituted of a porous material.

10. The device of claim 1, wherein
a peptide is attached to a surface of the adsorbing material.

11. A molecule detecting method, comprising:
detecting a first molecular group in a gaseous sample by means of a first detecting element;
passing the gaseous sample through an adsorbing material configured to adsorb at least the first molecular group;
detecting a second molecular group having a concentration higher than the first molecular group in the gaseous sample after being passed through the adsorbing material by means of a second detecting element; and
determining a type of the gaseous sample from results of detecting the first molecular group and detecting the second molecular group.

12. The method of claim 11, wherein
in determining the type of the gaseous sample, the type of the gaseous sample is determined by using an operation result to be obtained by subtracting a measurement result of detecting the second molecular group from a measurement result of detecting the first molecular group.

13. The method of claim 11, wherein
the first detecting element includes a first probe configured to combine with the first molecular group, and
the second detecting element includes a second probe configured to combine with the second molecular group.

14. The method of claim 13, wherein
the first probe and/or the second probe are constituted of an organic substance.

15. The method of claim 14, wherein
the first probe and/or the second probe are constituted of a peptide.

16. The method of claim 13, wherein
detecting the first molecular group is carried out by using a plurality of first detecting elements each including first probes of types different from each other,
detecting the second molecular group is carried out by using a plurality of second detecting elements each including second probes of types different from each other, and
determining the type of the gaseous sample includes determining the type of the gaseous sample by using a first pattern formed by adding up measurement results from the plurality of first detecting elements and a second pattern formed by adding up measurement results from the plurality of second detecting elements.

17. The method of claim 13, wherein
the first detecting element includes a graphene film possessing a surface on which the first probe is fixed, a source electrode connected to one end of the graphene film, and a drain electrode connected to the other end of the graphene film, and
the second detecting element includes a graphene film possessing a surface on which the second probe is fixed, a source electrode connected to one end of the graphene film, and a drain electrode connected to the other end of the graphene film.

18. The method of claim 11, wherein
the adsorbing material is constituted of a porous material.

19. The method of claim 11, wherein
a peptide is attached to a surface of the adsorbing material.
